(19) United States
(12) Patent Application Publication
(10) Pub. No.: US 2020/0254203 A1
(43) Pub. Date: Aug. 13, 2020

(54) TRACHEOSTOMY PLATE DECORATIVE COVER

(71) Applicant: Nikischia Lawrence, Richard, GA (US)

(72) Inventor: Nikischia Lawrence, Richard, GA (US)

(21) Appl. No.: 16/790,725

(22) Filed: Feb. 13, 2020

Related U.S. Application Data
(60) Provisional application No. 62/805,297, filed on Feb. 13, 2019.

(51) Int. Cl. A61M 16/04

(52) U.S. Cl. CPC A61M 16/047; A61M 2205/59; A61M 16/0497

(57) ABSTRACT

Implementations of a tracheostomy plate decorative cover are provided. In some implementations, the tracheostomy plate decorative cover comprises a front surface, a back surface, a left side, a right side, a top side, a bottom side, a tracheostomy tube opening, a first neck strap opening, and a second neck strap opening.

In some implementations, a method for using the tracheostomy plate decorative cover comprises positioning and/or attaching the tracheostomy plate decorative cover to a tracheostomy plate. In some implementations, the method may also comprise attaching the tracheostomy plate to a tracheostomy tube. In some implementations, the method may also comprise attaching a neck strap to the tracheostomy plate.

### CROSS REFERENCE TO RELATED APPLICATION

This application claims the benefit of U.S. Patent Application Ser. No. 62/805,297, which was filed on Feb. 13, 2019, and is incorporated herein by reference in its entirety.

### TECHNICAL FIELD

This disclosure relates to implementations of a decorative cover for a tracheostomy plate.

### BACKGROUND

As shown in FIG. 1, a tracheostomy is a surgically formed opening into the trachea (or windpipe) through a person's neck. The tracheostomy is usually made to provide an alternate opening instead of the nose or mouth for breathing air. A tracheostomy tube (or “trach tube”) is usually positioned through the tracheostomy to provide a breathing airway and medical access to the trachea and lungs.

As shown in FIG. 2, a tracheostomy plate (or “trach plate”), also referred to as a flange, neck plate, or faceplate, attaches to the trach tube. The trach plate attaches to the near (or proximal) end of the trach tube so that the trach plate rests against the person's neck who has the tracheostomy. The trach plate, so positioned and attached, prevents the trach tube from inserting further than intended into the trachea through the tracheostomy.

Furthermore, as also shown in FIG. 2, a neck strap or similar support attaches to the trach plate, for example, through an opening on each side of the trach plate. The neck strap, attached to the trach plate from around the person's neck, holds the trach plate in position to further support and minimize the movement of the trach tube positioned in the tracheostomy.

However, the trach plate is unsightly, e.g. utilitarian, and also does not reduce the unsightliness of the attached trach tube and neck strap, which may cause the wearer to be undesirably self conscious.

### BRIEF DESCRIPTION OF THE DRAWINGS

FIG. 1 illustrates a tracheostomy tube positioned in a tracheostomy.

FIG. 2 illustrates a tracheostomy plate attached to a neck strap and a tracheostomy tube positioned in a tracheostomy.

FIG. 3 illustrates a line representation of an implementation of an example tracheostomy plate decorative cover according to the present disclosure.

FIG. 4 illustrates the example tracheostomy plate decorative cover attached to a tracheostomy plate according to the present disclosure.

FIG. 5 illustrates the example tracheostomy plate decorative cover attached to a tracheostomy plate that is attached to a tracheostomy tube and a neck strap according to the present disclosure.

FIG. 6 illustrates a line representation of a plurality of the example tracheostomy plate decorative covers grouped in a sheet for bulk distribution and/or for usage according to the present disclosure.

FIG. 7 illustrates an exploded side cross-section view of the example tracheostomy plate decorative cover attached to the tracheostomy plate shown in FIG. 4.

### DETAILED DESCRIPTION

Implementations of a tracheostomy plate decorative cover are provided. In some implementations, the tracheostomy plate decorative cover comprises a front surface and a back surface. In some implementations, the tracheostomy plate decorative cover also comprises a left side, a right side, a top side, and a bottom side. In some implementations, the tracheostomy plate decorative cover also comprises a tracheostomy tube opening, a first neck strap opening, and a second neck strap opening.

In some implementations, the tracheostomy plate decorative cover is configured to attach to a tracheostomy plate. In some implementations, the tracheostomy plate decorative cover is configured to removably attach to a tracheostomy plate.

In some implementations, the tracheostomy plate decorative cover is configured to attach to a tracheostomy plate that is attached to a tracheostomy tube positioned in a tracheostomy.

In some implementations, the tracheostomy plate decorative cover is configured to attach to a tracheostomy plate that is also attached to a neck strap or similar support.

In some implementations, the tracheostomy plate decorative cover is configured to cover a tracheostomy plate. For example, in some implementations, the tracheostomy plate decorative cover is configured to cover the outward facing surface of a tracheostomy plate while the tracheostomy plate is attached to a tracheostomy tube positioned in a tracheostomy.

In some implementations, the tracheostomy plate decorative cover is configured to display a decorative image while attached to a tracheostomy plate.

In some implementations, the tracheostomy plate decorative cover is configured to decorate the appearance of a tracheostomy plate while the tracheostomy plate decorative cover is attached to the tracheostomy plate.

In some implementations, the tracheostomy plate decorative cover is configured to decorate the appearance of a tracheostomy plate attached to a tracheostomy tube positioned in a tracheostomy while the tracheostomy plate decorative cover is attached to the tracheostomy plate.

In some implementations, the tracheostomy plate decorative cover is configured to decorate the appearance of a tracheostomy plate that is attached to a tracheostomy tube positioned in a tracheostomy and to a neck strap or similar support while the tracheostomy plate decorative cover is attached to the tracheostomy plate.

In some implementations, the tracheostomy plate decorative cover is configured to add a decorative appearance that reduces the unsightliness of a tracheostomy tube positioned in a tracheostomy that is attached to the tracheostomy plate while the tracheostomy plate decorative cover is attached to the tracheostomy plate.

In some implementations, the tracheostomy plate decorative cover is configured to add a decorative appearance that reduces the unsightliness of a tracheostomy tube positioned in a tracheostomy and of a neck strap or similar support that are attached to the tracheostomy plate while the tracheostomy plate decorative cover is attached to the tracheostomy plate.

In some implementations, a method for using the tracheostomy plate decorative cover comprises positioning and/or attaching the tracheostomy plate decorative cover to a tracheostomy plate.

In some implementations, the method may also comprise attaching the tracheostomy plate, with the positioned and/or attached tracheostomy plate decorative cover, to a tracheostomy tube that is positioned in a tracheostomy.

In some implementations, the method may also comprise attaching a neck strap or similar support to the tracheostomy plate with the positioned and/or attached tracheostomy plate decorative cover while the tracheostomy plate is attached to the tracheostomy tube.

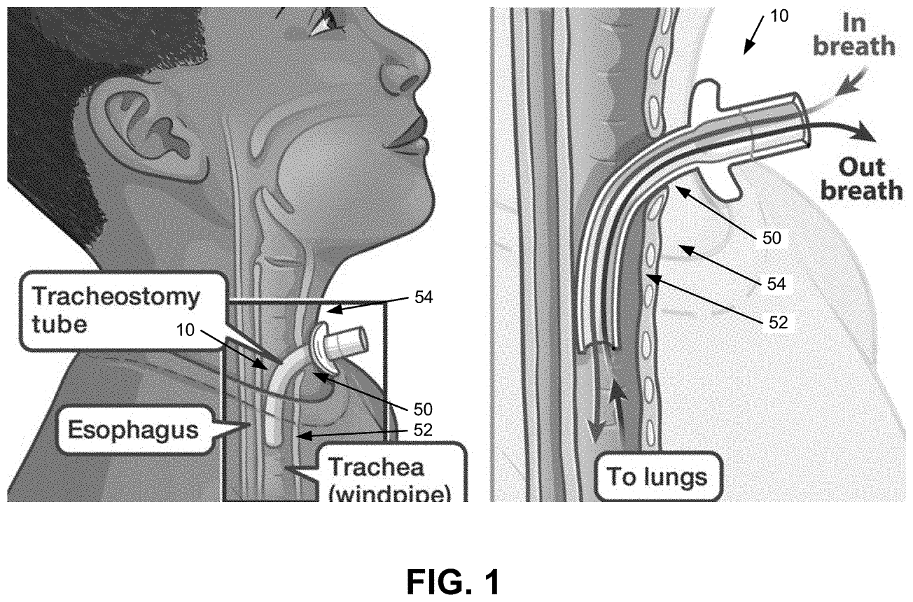

FIG. 1 illustrates a tracheostomy tube 10 positioned in a tracheostomy 50. As shown in FIG. 1, the tracheostomy 50 is a surgically formed opening into the trachea (or windpipe) 52 through a person's neck 54. The tracheostomy 50 is usually made to provide an alternate opening instead of the nose or mouth for breathing air. The tracheostomy tube (or “trach tube”) 10 is usually positioned through the tracheostomy 50 to provide a breathing airway and medical access to the trachea 52 and lungs (not shown).

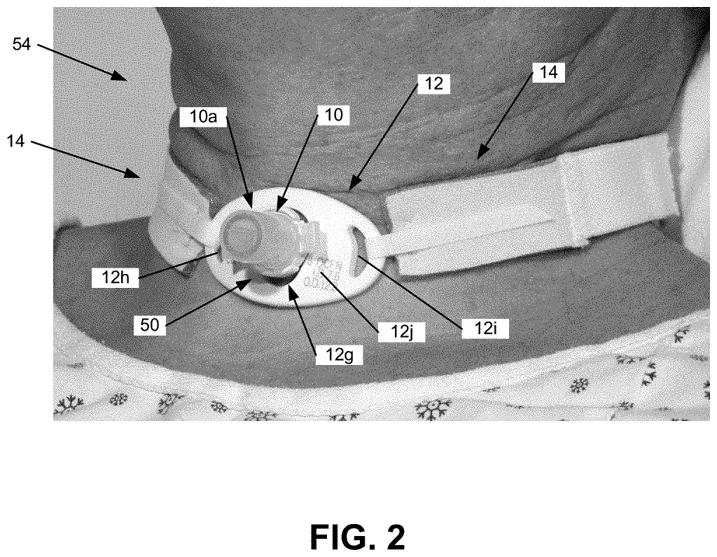

FIG. 2 illustrates a tracheostomy plate 12 attached to a neck strap 14 and a trach tube 10 positioned in a tracheostomy 50. As shown in FIG. 2, the tracheostomy plate (or “trach plate”) 12, also referred to as a flange, neck plate, or faceplate, attaches to the trach tube 10. The trach plate 12 attaches to the near (or proximal) end 10a of the trach tube 10 so that the trach plate 12 rests against the person's neck 54 who has the tracheostomy 50. Also, the trach tube 10 positions on and/or attaches to the trach plate 12 through the tube opening 12g.

The trach plate 12, so positioned and/or attached, prevents the trach tube 10 from inserting further than intended into the trachea 52 through the tracheostomy 50. An outward facing surface 12j of the trach plate 12 is exposed when the trach plate 12 is so positioned and attached.

Furthermore, as also shown in FIG. 2, the neck strap 14 or similar support attaches to the trach plate 12, for example, through an opening 12h, 12i on each side respectively of the trach plate 12. The neck strap 14, attached to the trach plate 12 from around the person's neck 54, holds the trach plate 12 in position to further support and minimize the movement of the trach tube 10 positioned in the tracheostomy 50.

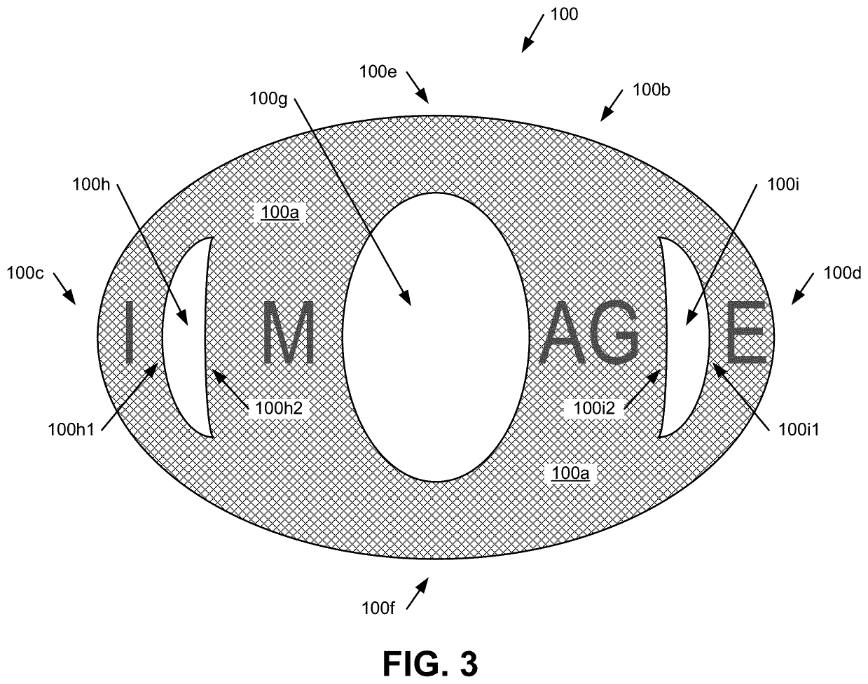

FIG. 3 illustrates a line representation of an implementation of an example tracheostomy plate decorative cover 100 according to the present disclosure. In some implementations, the tracheostomy plate decorative cover 100 comprises a front surface 100a, a back surface 100b, a left side 100c, a right side 100d, a top side 100e, a bottom side 100f, a tracheostomy tube opening 100g, a first neck strap opening 100h, and a second neck strap opening 100i.

In some implementations, the tracheostomy plate decorative cover 100 is flat. For example, in some implementations, the depth or thickness of the tracheostomy plate decorative cover 100 between the front surface 100a and the back surface 100b is very thin. In some implementations, the thickness of the tracheostomy plate decorative cover 100 is similar to the thickness of a sheet of paper, plastic, or sticker.

For example, in some implementations, the thickness of the tracheostomy plate decorative cover 100 may be 0.006 to 0.007 inches. In some implementations, the thickness of the tracheostomy plate decorative cover 100 may be less than or equal to 0.006 inches. In some implementations, the thickness of the tracheostomy plate decorative cover 100 may be greater than or equal to 0.007 inches.

In some implementations, the thickness of the material may be 0.0169 inches. In some implementations, the thickness of the material may be less than 0.0169 inches. In some implementations, the thickness of the material may be greater than 0.0169 inches. In some implementations, the thickness of the material may less than or equal to 0.007 inches. In some implementations, the thickness of the material may be between 0.006 inches to 0.007 inches.

In some implementations, the tracheostomy plate decorative cover 100 is oval shaped. For example, in some implementations, the tracheostomy plate decorative cover 100 is curved around the edge of the tracheostomy plate decorative cover 100 along the top side 100e, the left side 100c, the bottom side 100f, and the right side 100d. Furthermore, in some implementations, the length of the tracheostomy plate decorative cover 100 between the left side 100c and the right side 100d is greater than the width of the tracheostomy plate decorative cover 100 between the top side 100e and the bottom side 100f.

In some implementations, the tracheostomy plate decorative cover 100 may have any other suitable shape and/or dimensional proportions.

In some implementations, the extents of the front surface 100a and of the back surface 100b of the tracheostomy plate decorative cover 100 are defined by the sides 100c, 100d, 100e, 100f and the openings 100g, 100h, 100i of the tracheostomy plate decorative cover 100.

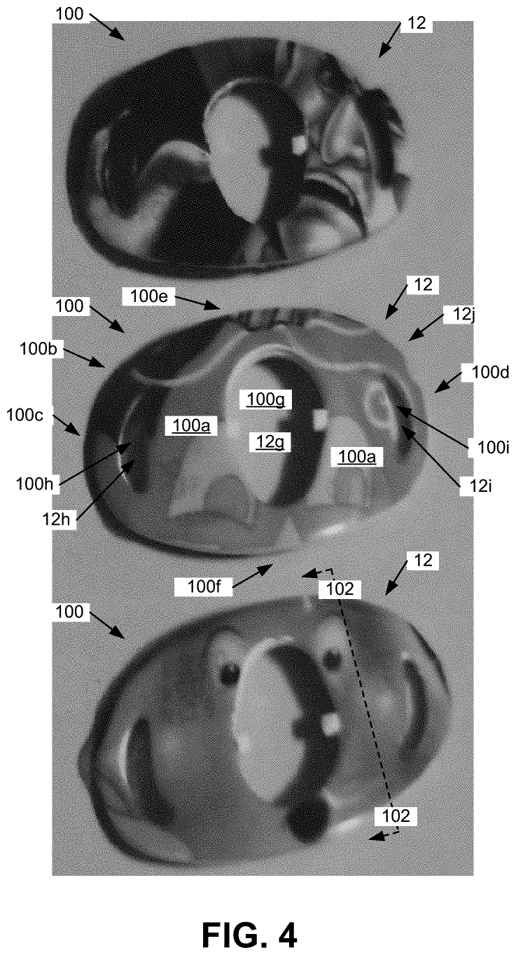

In some implementations, the front surface 100a of the tracheostomy plate decorative cover 100 is configured to display a decorative image. In some implementations, the front surface 100a is configured to display a decorative image that can decorate the appearance of a tracheostomy plate, such as the trach plate 12 shown in FIG. 4, and/or other attached tracheostomy components, as described below.

In some implementations, the decorative image may be a graphic image, such as the example images displayed by the front surfaces 100a shown in FIG. 4. For example, in some implementations, the decorative image displayed by the front surface 100 may be a cartoon or caricature image. In some implementations, the decorative image displayed by the front surface 100 may be a pattern or graphic design.

In some implementations, the decorative image displayed by the front surface 100 may comprise animate and/or inanimate content. In some implementations, the decorative image displayed by the front surface 100 may comprise one or more colors. In some implementations, the decorative image displayed by the front surface 100 may comprise one or more other graphic properties.

In some implementations, the decorative image displayed by the front surface 100 may be any other suitable image(s) that can provide a decorative appearance.

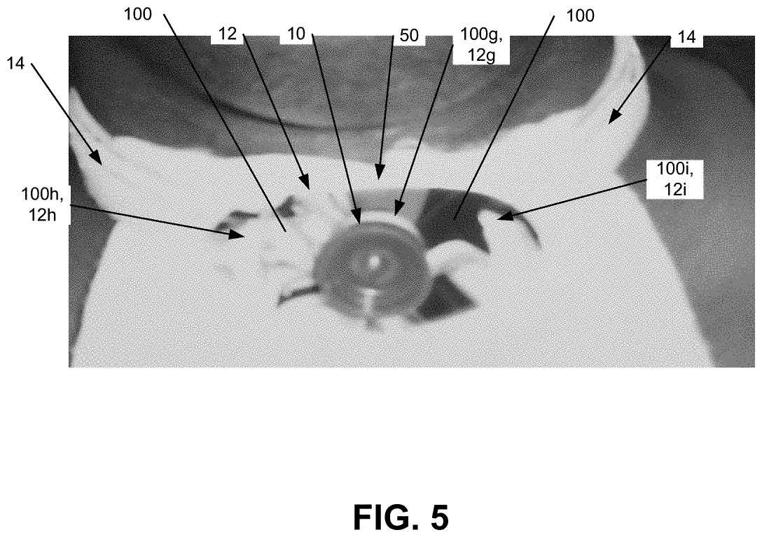

In some implementations, the back surface 100b of the tracheostomy plate decorative cover 100 is configured to position on a tracheostomy plate, such as the trach plate 12 shown in FIG. 4. In some implementations, the back surface 100b is configured to position on the outward facing surface of a tracheostomy plate that is attached to a tracheostomy tube positioned in a tracheostomy, such as the trach tube 10 and tracheostomy 50 shown in FIG. 5 and the outward facing surface 12j shown in FIG. 2.

In some implementations, the back surface 100b is configured to adhere or otherwise attach to the tracheostomy plate. In some implementations, the back surface 100b is configured to removably adhere or otherwise attach to the tracheostomy plate.

For example, in some implementations, the back surface 100b may be configured to cling to the outward facing surface of the tracheostomy plate. In some implementations, the back surface 100b may be configured to cling to the outward facing surface by static charge, static cling, suction, coadhesive forces, etc. and without glue or other adhesive component so that the tracheostomy plate decorative cover 100 can be easily attached and removed from the tracheostomy plate.

In some implementations, the back surface 100b may be configured to removably adhere to the outward facing surface of the tracheostomy plate by a natural contact-adhesion characteristic of the material that composes the tracheostomy plate decorative cover 100.

In some implementations, the back surface 100b may be configured to adhere or otherwise attach to the tracheostomy plate by the support of and/or attachment to other tracheostomy components, such as through the openings 100g, 100h, 100i of the tracheostomy plate decorative cover 100. For example, in some implementations, the back surface 100b may be configured to adhere or otherwise attach to the tracheostomy plate by the support of and/or attachment to the tracheostomy tube and/or a neck strap or similar support, such as the neck strap 14 shown in FIG. 5.

In some implementations, the back surface 100b may be configured to adhere or otherwise attach to the tracheostomy plate, including removably, by any other suitable way. For example, in some implementations, the back surface 100b may be configured to removably adhere or otherwise attach to the tracheostomy plate by a reusable adhesive on the back surface 100b.

In some implementations, the tracheostomy plate decorative cover 100 is made from a thin, flexible, pliable, etc. plastic material having the features described above. For example, in some implementations, the material may be a vinyl material.

In some implementations, the tracheostomy tube opening 100g of the tracheostomy plate decorative cover 100 is the same or substantially similar to the tube opening of the tracheostomy plate to which the tracheostomy plate decorative cover 100 is to be attached, such as the tube opening 12g of the tracheostomy plate 12 shown in FIG. 2. For example, in some implementations, the tracheostomy tube opening 100g is oval shaped such that the tracheostomy tube opening 100g is curved around the edge of the tracheostomy tube opening 100g along the sides of the tracheostomy tube opening 100g. Furthermore, in some implementations, the width of the tracheostomy tube opening 100g that extends in between the top side 100e and bottom side 100f of the tracheostomy plate decorative cover 100 is greater than the length of the tracheostomy tube opening 100g that extends in between the left side 100c and the right side 100d of the tracheostomy plate decorative cover 100.

In some implementations, the tracheostomy tube opening 100g is configured to allow a tracheostomy tube to position through the tracheostomy tube opening 100g while the tracheostomy plate decorative cover 100 is positioned on and/or attached to the tracheostomy plate. In some implementations, the tracheostomy tube opening 100g is configured to allow the tracheostomy tube to position through the tracheostomy tube opening 100g in the same or similar manner that the tracheostomy tube positions through the tube opening of the tracheostomy plate.

In some implementations, the neck strap openings 100h, 100i of the tracheostomy plate decorative cover 100 respectively comprise curved sides 100h1, 100i1 and substantially straight (and/or slightly curved) sides 100h2, 100i2.

In some implementations, the neck strap openings 100h, 100i of the tracheostomy plate decorative cover 100 are the same or substantially similar respectively to the strap openings of the tracheostomy plate to which the tracheostomy plate decorative cover 100 is to be attached, such as the strap openings 12h, 12i of the tracheostomy plate 12 shown in FIG. 2.

For example, in some implementations, the first neck strap opening 100h is crescent shaped such that the sides 100h1, 100h2 extend in between the top side 100e and the bottom side 100f of the tracheostomy plate decorative cover 100 with the curved side 100h1 adjacent to the left side 100c of the tracheostomy plate decorative cover 100. Similarly, in some implementations, the second neck strap opening 100i is crescent shaped such that the sides 100i1, 100i2 extend in between the top side 100e and bottom side 100f of the tracheostomy plate decorative cover 100 with the curved side 100i1 adjacent to the right side 100d of the tracheostomy plate decorative cover 100.

In some implementations, the neck strap openings 100h, 100i are configured to receive a neck strap or similar support, such as the neck strap 14 shown in FIG. 5, respectively through each neck strap opening 100h, 100i while the tracheostomy plate decorative cover 100 is positioned on and/or attached to the tracheostomy plate. In some implementations, the neck strap openings 100h, 100i are configured to receive the neck strap or similar support respectively in the same or similar manner that the strap openings of the tracheostomy plate receive the neck strap or similar support respectively through each strap opening.

As shown in FIG. 4, which is described below, in some implementations, the extents of the tracheostomy plate decorative cover 100 are configured to be the same or substantially similar to the extents of the tracheostomy plate to which the tracheostomy plate decorative cover 100 is configured to be positioned on and/or attached. That is, in some implementations, the sides 100c, 100d, 100e, 100f of the tracheostomy plate decorative cover 100 are configured to align or substantially align with the corresponding sides of the tracheostomy plate when the tracheostomy plate decorative cover 100 is positioned on and/or attached to the tracheostomy plate. Similarly, in some implementations, the openings 100g, 100h, 100i of the tracheostomy plate decorative cover 100 are configured to align or substantially align with the corresponding openings 12g, 12h, 12i of the tracheostomy plate when the tracheostomy plate decorative cover 100 is positioned on and/or attached to the tracheostomy plate.

In some implementations, the tracheostomy plate decorative cover 100 is configured to attach to a tracheostomy plate, such as the trach plate 12 shown in FIG. 4. In some implementations, the tracheostomy plate decorative cover 100 is configured to removably attach to the tracheostomy plate.

In some implementations, the tracheostomy plate decorative cover 100 is configured to attach to a tracheostomy plate that is attached to a tracheostomy tube positioned in a tracheostomy, such as the trach tube 10 positioned in the tracheostomy 50 shown in FIG. 5.

In some implementations, the tracheostomy plate decorative cover 100 is configured to attach to a tracheostomy plate that is also attached to a neck strap or similar support, such as the neck strap 14 shown in FIG. 5.

In some implementations, the tracheostomy plate decorative cover 100 is configured to cover a tracheostomy plate. For example, in some implementations, the tracheostomy plate decorative cover 100 is configured to cover the outward facing surface of a tracheostomy plate, such as the outward facing surface 12j shown in FIG. 2, while the tracheostomy plate is attached to a tracheostomy tube positioned in a tracheostomy.

In some implementations, the tracheostomy plate decorative cover 100 is configured to display a decorative image while attached to a tracheostomy plate.

In some implementations, the tracheostomy plate decorative cover 100 is configured to decorate the appearance of a tracheostomy plate while the tracheostomy plate decorative cover 100 is attached to the tracheostomy plate.

In some implementations, the tracheostomy plate decorative cover 100 is configured to decorate the appearance of a tracheostomy plate attached to a tracheostomy tube positioned in a tracheostomy while the tracheostomy plate decorative cover 100 is attached to the tracheostomy plate.

In some implementations, the tracheostomy plate decorative cover 100 is configured to decorate the appearance of a tracheostomy plate attached to a tracheostomy tube positioned in a tracheostomy and to a neck strap or similar support while the tracheostomy plate decorative cover 100 is attached to the tracheostomy plate.

In some implementations, the tracheostomy plate decorative cover 100 is configured to add a decorative appearance that reduces the unsightliness of a tracheostomy tube positioned in a tracheostomy that is attached to the tracheostomy plate while the tracheostomy plate decorative cover 100 is attached to the tracheostomy plate.

In some implementations, the tracheostomy plate decorative cover 100 is configured to add a decorative appearance that reduces the unsightliness of a tracheostomy tube positioned in a tracheostomy and of a neck strap or similar support that are attached to the tracheostomy plate while the tracheostomy plate decorative cover 100 is attached to the tracheostomy plate.

FIG. 4 illustrates the example tracheostomy plate decorative cover 100 attached to a tracheostomy plate 12 according to the present disclosure.

In some implementations, the tracheostomy plate decorative cover 100 is positioned on and/or attached to the tracheostomy plate 12, such as by one or more of the ways described above. For example, in some implementations, the back surface 100b of the tracheostomy plate decorative cover 100 is adhered or otherwise attached to the outward facing surface 12j of the tracheostomy plate 12.

In some implementations, the tracheostomy plate decorative cover 100 is aligned or substantially aligned with the tracheostomy plate 12. For example, in some implementations, the sides 100c, 100d, 100e, 100f of the tracheostomy plate decorative cover 100 are aligned or substantially aligned with the corresponding sides of the tracheostomy plate 12. Similarly, in some implementations, the openings 100g, 100h, 100i of the tracheostomy plate decorative cover 100 are aligned or substantially aligned with the corresponding openings 12g, 12h, 12i of the tracheostomy plate 12.

In some implementations, the tracheostomy plate decorative cover 100 covers the tracheostomy plate 12. For example, in some implementations, the tracheostomy plate decorative cover 100 covers the outward facing surface 12j of the tracheostomy plate 12.

In some implementations, the tracheostomy plate decorative cover 100 displays a decorative image while attached to the tracheostomy plate 12. For example, in some implementations, the front surface 100a of the tracheostomy plate decorative cover 100 displays a decorative graphic image.

In some implementations, the tracheostomy plate decorative cover 100 decorates the appearance of the tracheostomy plate 12. For example, in some implementations, the tracheostomy plate decorative cover 100 decorates the appearance of the tracheostomy plate 12 by the displayed decorative image.

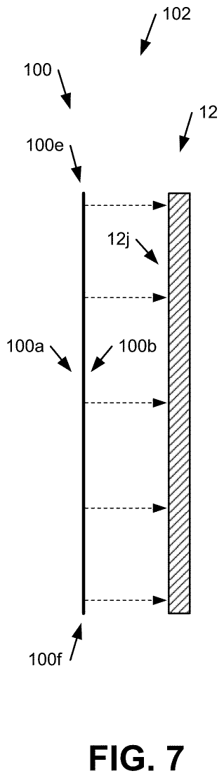

An exploded side cross-section view 102 of the tracheostomy plate decorative cover 100 attached to the tracheostomy plate 12, as indicated in FIG. 4, is described below for FIG. 7.

FIG. 5 illustrates the example tracheostomy plate decorative cover 100 attached to a tracheostomy plate 12 that is attached to a tracheostomy tube 10 and a neck strap 14 according to the present disclosure.

In some implementations, the tracheostomy plate decorative cover 100 is positioned on and/or attached to the tracheostomy plate 12 as described above for FIG. 4.

In some implementations, the tracheostomy plate decorative cover 100 is aligned or substantially aligned with the tracheostomy plate 12 as described above for FIG. 4.

In some implementations, the tracheostomy plate decorative cover 100 covers the tracheostomy plate 12 as described above for FIG. 4.

In some implementations, the tracheostomy tube 10 is positioned in a tracheostomy 50.

In some implementations, the tracheostomy tube 10 is positioned in the tracheostomy 50 through the tracheostomy tube opening 100g of the tracheostomy plate decorative cover 100, which is aligned with the tube opening 12g of the tracheostomy plate 12.

In some implementations, the neck strap 14 or similar support is attached to the tracheostomy plate 12 through the neck strap openings 100h, 100i of the tracheostomy plate decorative cover 100, which are aligned respectively with the strap openings 12h, 12i of the tracheostomy plate 12.

In some implementations, the tracheostomy plate decorative cover 100 displays a decorative image while attached to the tracheostomy plate 12 as described above for FIG. 4.

In some implementations, the tracheostomy plate decorative cover 100 decorates the appearance of the tracheostomy plate 12 as described above for FIG. 4.

In some implementations, the tracheostomy plate decorative cover 100 adds a decorative appearance that reduces the unsightliness of the tracheostomy tube 10.

In some implementations, the tracheostomy plate decorative cover 100 adds a decorative appearance that reduces the unsightliness of the neck strap 14 or similar support.

In some implementations, the tracheostomy plate decorative cover 100 adds a decorative appearance that reduces the unsightliness of the tracheostomy tube 10 and the neck strap 14 or similar support.

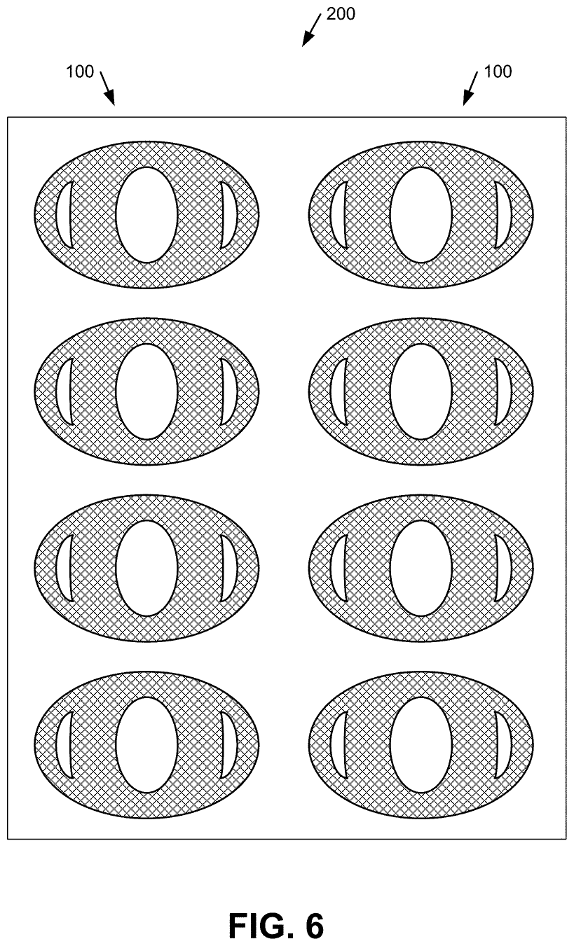

FIG. 6 illustrates a line representation of a plurality of the example tracheostomy plate decorative covers 100 grouped in a sheet 200 for bulk distribution and/or for usage according to the present disclosure.

In some implementations, the tracheostomy plate decorative cover 100 is configured to be grouped on the sheet 200 for bulk distribution and/or for usage.

In some implementations, the tracheostomy plate decorative cover 100 is configured to be grouped on the sheet 200 in any suitable plurality, such as two or more.

In some implementations, the tracheostomy plate decorative cover 100 is configured to be grouped on the sheet 200 in any suitable pattern, such as horizontally in rows and/or vertically in columns.

In some implementations, the tracheostomy plate decorative cover 100 is configured to be removed or otherwise separated from the sheet 200 as needed to be positioned on and/or attached to a tracheostomy plate as discussed above.

In some implementations, the tracheostomy plate decorative cover 100 may also be configured to be replaced or otherwise reattached to the sheet 200, for example to later reuse the tracheostomy plate decorative cover 100 for another application to a tracheostomy plate.

In some implementations, the tracheostomy plate decorative cover 100 is configured to adhere or otherwise attach to the sheet 200 in one of the same or similar ways as described above with respect to the attachment of the back surface 100b of the tracheostomy plate decorative cover 100 to a tracheostomy plate. For example, in some implementations, the tracheostomy plate decorative cover 100 may be configured to attach to the sheet 200 by a reusable adhesive.

In some implementations, the tracheostomy plate decorative cover 100 may be configured to adhere or otherwise attach to the sheet 200 in any other suitable way.

In some implementations, the sheet 200 may be similar to a sheet of paper. In some implementations, the sheet 200 may have any other suitable configuration.

In some implementations, the tracheostomy plate decorative cover 100 may be configured to be grouped in any other suitable configuration. For example, in some implementations, the tracheostomy plate decorative cover 100 may be configured to be grouped in a strip or a roll (not shown).

FIG. 7 illustrates an exploded side cross-section view 102 of the example tracheostomy plate decorative cover 100 attached to the tracheostomy plate 12 shown in FIG. 4, which is described above.

In some implementations, the exploded side cross-section view 102 includes the front surface 100a, the back surface 100b, the top side 100e, and the bottom side 100f of the tracheostomy plate decorative cover 100.

In some implementations, the exploded side cross-section view 102 also includes the outward facing surface 12j of the tracheostomy plate 12.

As shown in FIG. 7, in some implementations, the tracheostomy plate decorative cover 100 is relatively thinner in thickness, i.e. between the front surface 100a and the back surface 100b, than the thickness of the tracheostomy plate 12.

In some implementations, the tracheostomy plate decorative cover 100 comprises any suitable dimensions, such as the example dimensional proportions described above.

In some implementations, the tracheostomy plate decorative cover 100 is composed of any suitable materials, such as the example materials described above.

In some implementations, the tracheostomy plate decorative cover 100 can have any suitable appearance, such as the decorative image features described above.

In some implementations, an example method of using the tracheostomy plate decorative cover 100, such as shown by FIGS. 4 and 5, comprises positioning and/or attaching the tracheostomy plate decorative cover 100 to a tracheostomy plate, such as the tracheostomy plate 12 described above.

In some implementations, the method may also comprise attaching the tracheostomy plate, with the positioned and/or attached tracheostomy plate decorative cover 100, to a tracheostomy tube that is positioned in a tracheostomy.

In some implementations, the method may also comprise attaching a neck strap or similar support to the tracheostomy plate with the positioned and/or attached tracheostomy plate decorative cover 100 while attached to the tracheostomy tube.

In some implementations, the tracheostomy plate decorative cover 100 is positioned on and/or attached to the tracheostomy plate by one or more of the ways described above.

In some implementations, the tracheostomy plate decorative cover 100 is positioned on and/or attached to the tracheostomy plate so that the tracheostomy plate decorative cover 100 aligns or substantially aligns with the tracheostomy plate as described above.

In some implementations, the tracheostomy plate decorative cover 100 is positioned on and/or attached to the tracheostomy plate so that the tracheostomy plate decorative cover 100 covers the tracheostomy plate as described above.

In some implementations, the tracheostomy plate decorative cover 100 is positioned on and/or attached to the tracheostomy plate so that the tracheostomy plate decorative cover 100 displays a decorative image as described above.

In some implementations, the tracheostomy plate decorative cover 100 is positioned on and/or attached to the tracheostomy plate so that the tracheostomy plate decorative cover 100 decorates the appearance of the tracheostomy plate as described above.

In some implementations, the tracheostomy plate decorative cover 100 is positioned on and/or attached to the tracheostomy plate so that an attached tracheostomy tube positions in a tracheostomy through the tracheostomy tube opening 100g of the tracheostomy plate decorative cover 100 as described above.

In some implementations, the tracheostomy plate decorative cover 100 is positioned on and/or attached to the tracheostomy plate so that a neck strap or similar support attaches to the tracheostomy plate through the neck strap openings 100h, 100i of the tracheostomy plate decorative cover 100 respectively as described above.

In some implementations, the tracheostomy plate decorative cover 100 is positioned on and/or attached to the tracheostomy plate so that the tracheostomy plate decorative cover 100 adds a decorative appearance that reduces the unsightliness of the attached tracheostomy tube and/or the neck strap or similar support as described above.

In some implementations, the method of using the tracheostomy plate decorative cover further comprises obtaining the tracheostomy plate decorative cover 100 from a sheet on which a plurality of the tracheostomy plate decorative covers 100 are grouped for bulk distribution and/or for usage, such as the sheet 200 described above for FIG. 6.

In some implementations, the method also comprises replacing the tracheostomy plate decorative cover 100 to the sheet, for example to later reuse the tracheostomy plate decorative cover 100 for another application to a tracheostomy plate.

In some implementations, the method of using the tracheostomy plate decorative cover further comprises removing the tracheostomy plate decorative cover 100 from the tracheostomy plate.

In some implementations, the method also comprises positioning and/or attaching another tracheostomy plate decorative cover 100 to the tracheostomy plate, e.g. that displays a different decorative image.

The figures, including photographs and drawings, comprised herewith may represent one or more implementations of the tracheostomy plate decorative cover.

Details shown in the figures, such as dimensions, descriptions, etc., are exemplary, and there may be implementations of other suitable details according to the present disclosure.

Reference throughout this specification to “an embodiment” or “implementation” or words of similar import means that a particular described feature, structure, or characteristic is comprised in at least one embodiment of the present invention. Thus, the phrase “in some implementations” or a phrase of similar import in various places throughout this specification does not necessarily refer to the same embodiment.

Many modifications and other embodiments of the inventions set forth herein will come to mind to one skilled in the art to which these inventions pertain having the benefit of the teachings presented in the foregoing descriptions and the associated drawings.

The described features, structures, or characteristics may be combined in any suitable manner in one or more embodiments. In the above description, numerous specific details are provided for a thorough understanding of embodiments of the invention. One skilled in the relevant art will recognize, however, that embodiments of the invention can be practiced without one or more of the specific details, or with other methods, components, materials, etc. In other instances, well-known structures, materials, or operations may not be shown or described in detail.

While operations may be depicted in the drawings in a particular order, this should not be understood as requiring that such operations be performed in the particular order shown or in sequential order, or that all illustrated operations be performed, to achieve desirable results.

1. A tracheostomy plate decorative cover configured to cover a tracheostomy plate that attaches to a tracheostomy tube that positions in a tracheostomy, the decorative cover comprising a piece of material sized and shaped such that the decorative cover coextensively covers and corresponds to the openings of an outward facing surface of the tracheostomy plate when the tracheostomy plate is attached to the tracheostomy tube that is positioned in the tracheostomy, wherein:
the piece of material comprises a front surface and an opposite back surface defined by and extending between a left side and an opposite right side and a top side and an opposite bottom side;
the front surface is configured to display a decorative image that decorates the appearance of the outward facing surface of the tracheostomy plate when the decorative cover is positioned on the tracheostomy plate attached to the tracheostomy tube positioned in the tracheostomy; and
the back surface is configured to position on the outward facing surface of the tracheostomy plate such that the decorative cover is securable to the tracheostomy plate.

2. The tracheostomy plate decorative cover of claim 1, wherein the piece of material comprises a thin, pliable, plastic piece of material and the back surface is configured to removably adhere to the outward facing surface of the tracheostomy plate by cohesive forces without an adhesive.

3. The tracheostomy plate decorative cover of claim 1, wherein the back surface is configured to removably cling to the outward facing surface of the tracheostomy plate without an adhesive.

4. The tracheostomy plate decorative cover of claim 1, wherein the back surface is configured to removably adhere to the outward facing surface of the tracheostomy plate.

5. The tracheostomy plate decorative cover of claim 1, further comprising a tracheostomy tube opening, a first neck strap opening, and second neck strap opening each extending through the front surface and the back surface of the decorative cover and configured to correspond to a tracheostomy tube opening, a first neck strap opening, and second neck strap opening of the tracheostomy plate.

6. The tracheostomy plate decorative cover of claim 1, wherein the decorative cover is further configured to cover the tracheostomy plate that also attaches to a tracheostomy plate neck strap.

7. The tracheostomy plate decorative cover of claim 1, wherein the decorative image comprises a cartoon or caricature image.

8. The tracheostomy plate decorative cover of claim 1, wherein the decorative image comprises a pattern or graphic design.

9. The tracheostomy plate decorative cover of claim 1, wherein the decorative image comprises an animate or inanimate image.

10. The tracheostomy plate decorative cover of claim 1, wherein the decorative image comprises one or more colors.

11. The tracheostomy plate decorative cover of claim 1, wherein the piece of material comprises a flat, thin piece of material.

12. The tracheostomy plate decorative cover of claim 1, wherein the piece of material comprises an oval shaped piece of material.

13. The tracheostomy plate decorative cover of claim 1, wherein the decorative cover is further configured to be removably attached in a laid out group of the decorative covers on a sheet-like piece of material for storage and use of the decorative cover.

14. The tracheostomy plate decorative cover of claim 13, further comprising a plurality of the decorative covers removably attached to the sheet-like piece of material.

15. A method of using the tracheostomy plate decorative cover of claim 1, comprising positioning the back surface of the decorative cover on the outward facing surface of the tracheostomy plate such that the decorative cover is securable to the tracheostomy plate and the decorative image is displayed from the front surface of the decorative cover such that the decorative image decorates the appearance of the outward facing surface of the tracheostomy plate while attached to the tracheostomy tube positioned in the tracheostomy.

16. The method of claim 15, further comprising attaching the decorative cover to the tracheostomy plate.

17. The method of claim 15, further comprising attaching the tracheostomy plate, with the decorative cover positioned thereon, to the tracheostomy tube.

18. A method of using the tracheostomy plate decorative cover of claim 1, comprising:
positioning the back surface of the decorative cover on the outward facing surface of the tracheostomy plate such that the decorative cover is securable to the tracheostomy plate and the decorative image is displayed from the front surface of the decorative cover such that the decorative image decorates the appearance of the outward facing surface of the tracheostomy plate while attached to the tracheostomy tube positioned in the tracheostomy; and
attaching the tracheostomy plate, with the decorative cover positioned thereon, to the tracheostomy tube positioned in the tracheostomy.

19. A method of using the tracheostomy plate decorative cover of claim 6, comprising:
positioning the back surface of the decorative cover on the outward facing surface of the tracheostomy plate such that the decorative cover is securable to the tracheostomy plate and the decorative image is displayed from the front surface of the decorative cover such that the decorative image decorates the appearance of the outward facing surface of the tracheostomy plate while attached to the tracheostomy tube positioned in the tracheostomy; and
attaching the tracheostomy plate neck strap to the tracheostomy plate.

20. A method of using the tracheostomy plate decorative cover of claim 5, comprising:
positioning the back surface of the decorative cover on the outward facing surface of the tracheostomy plate such that the decorative cover is securable to the tracheostomy plate and the decorative image is displayed from the front surface of the decorative cover such that the decorative image decorates the appearance of the outward facing surface of the tracheostomy plate while attached to the tracheostomy tube positioned in the tracheostomy;
attaching the tracheostomy plate, with the decorative cover positioned thereon, to the tracheostomy tube; and
attaching the tracheostomy plate neck strap to the tracheostomy plate.
